(19) United States
(12) Patent Application Publication
(10) Pub. No.: US 2026/0292413 A1
(43) Pub. Date: Sep. 24, 2026

(54) ACTIVE COIL DAMPING

(71) Applicant: Cochlear Limited, Macquarie University, NSW (AU)

(72) Inventors: Pieterjan POLFLIET, Londerzeel (BE); Werner MESKENS, Opwijk (BE); Philip SAUVAGE, Geel (BE)

(21) Appl. No.: 19/477,336

(22) PCT Filed: Apr. 29, 2024
(86) PCT No.: PCT/IB2024/054153

§ 371 Date: Oct. 21, 2025

Related U.S. Application Data
(60) Provisional application No. 63/500,335, filed on May 5, 2023.

(51) Int. Cl. H04R 25/00; H01Q 7/00

(52) U.S. Cl. CPC H04R 25/554; H01Q 7/00; H04R 2225/51; H04R 2225/67

(57) ABSTRACT

Presented hearing are techniques for data integrity enhancement for inductively coupled resonant tanks, in particular, the techniques presented here use an external damping switch to perform selective and active damping of an external coil (headpiece coil), and is a device comprising: a radio frequency (RF) transceiver; an external coil assembly including an external coil configured to be inductively coupled with an implantable coil to form a closely-coupled RF link; and an external damping switch circuit electrically connected with the external coil, wherein the external damping switch circuit is controlled according to a programmable on-time for damping activation, to provide selective active damping of the external coil during transmission of data on the closely-coupled RF link.

### BACKGROUND

### Field of the Invention

The present invention relates generally to damping of an external coil of, for example, an implantable medical device.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a device is provided. The device comprises a radio frequency (RF) transceiver; an external coil assembly including an external coil configured to be inductively coupled with an implantable coil to form a closely-coupled RF link; and an external damping switch circuit electrically connected with the external coil, wherein the external damping switch circuit is controlled according to a programmable on-time for damping activation, to provide selective active damping of the external coil during transmission of data on the closely-coupled RF link.

In another aspect, a device is provided comprising: a radio frequency (RF) transceiver including one or more first drivers and one or more second drivers; a first coil assembly configured to be inductively coupled to a second coil assembly to form a closely-coupled RF link; an external damping switch circuit electrically connected with the first coil assembly; and a damping controller configured to control the external damping switch circuit to selectively damp the first coil assembly based on a coupling factor between the first coil assembly and the second coil assembly.

In another aspect, a method is provided. The method comprises: transferring at least data from an external coil assembly to an implantable coil assembly over a closely-coupled radio frequency (RF) link using an on-off keying (OOK) data scheme comprised of one cells and zero cells; setting a programmable on-time for damping activation during the zero cells; and selectively damping the external coil assembly during the zero cells according to the programmable on-time using an external damping switch circuit electrically connected with the external coil assembly.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 1E is a schematic diagram illustrating a computing device with which aspects of the techniques presented herein can be implemented;

FIG. 2A is a circuit diagram for an external coil with an external damping switch circuit coupled thereto, according to an example embodiment;

FIG. 2B is a circuit diagram for an external coil with an external damping switch circuit coupled thereto, according to another example embodiment;

FIG. 3 is a circuit diagram for an external coil with an external damping switch circuit coupled thereto, according to another example embodiment;

FIG. 4 is a circuit diagram showing a damping controller that is configured to control the external damping switch circuit of FIGS. 2A-2B and 3, according to an example embodiment;

FIG. 5A is a timing diagram showing a 5CPC RF data stream for a monophasic driver output signal (for legacy devices);

FIG. 5B is a timing diagram showing a 5CPC RF data stream for an inverted monophasic driver output signal;

FIG. 5C is a timing diagram showing a 5CPC RF data stream for a biphasic driver output signal;

FIG. 6A shows graphical simulation results plotting modulation depth as a function of damping active time for various coupling factors (k), with an implant load of 6.25 kOhm;

FIG. 6B shows graphical simulation results plotting RF link efficiency as a function of damping active time for various coupling factors (k), with an implant load of 6.25 kOhm;

FIG. 6C shows graphical simulation results plotting modulation depth as a function of damping active time for various coupling factors (k), with an implant load of 3.125 kOhm;

FIG. 6D shows graphical simulation results 640 plotting RF link efficiency as a function of damping active time for various coupling factors (k), with an implant load of 3.125 kOhm;

FIG. 7 is a flowchart of a method for direct active damping with adjustable strength, according to an example embodiment; and

FIG. 8 is a schematic diagram illustrating a vestibular stimulator system with which aspects of the techniques presented herein can be implemented.

### DETAILED DESCRIPTION

### Overview

Presented herein are techniques for data integrity enhancement for inductively coupled resonant tanks. In particular, the techniques presented herein use an external damping switch circuit to perform selective and active damping of an external coil (headpiece coil). For example, in certain embodiments, an adjustable damping mechanism is provided to set the moment of activation (programmable on-time) of the damping (e.g., immediately or after some delay from the end of a one cell, during a zero cell), thereby adjusting/controlling the data integrity level (and damping strength) and the link efficiency.

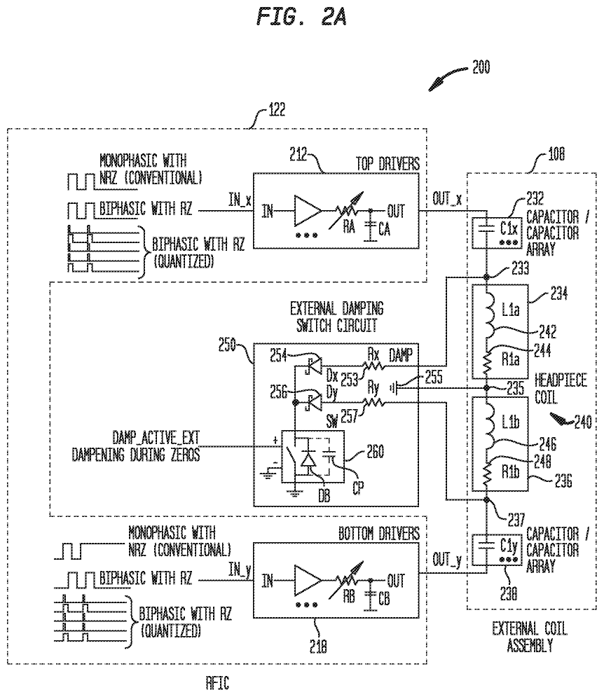

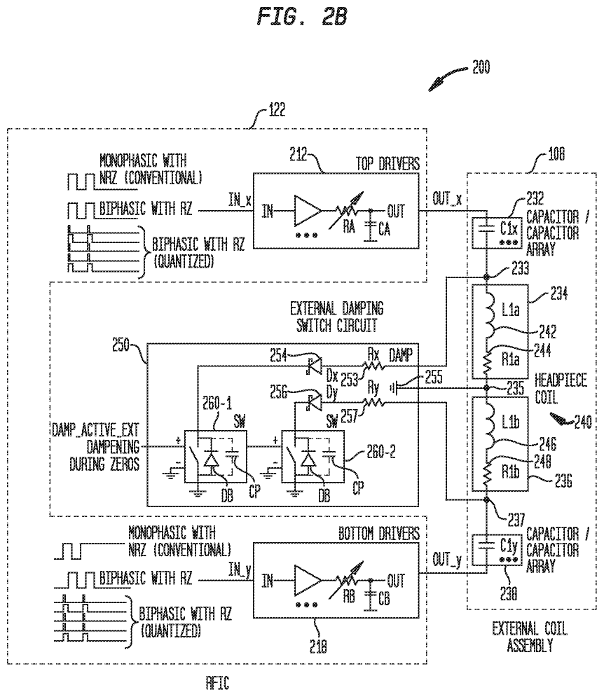

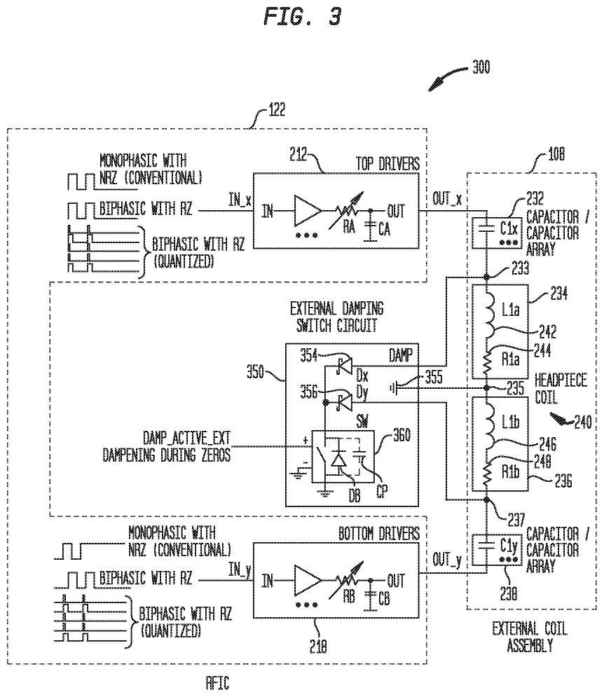

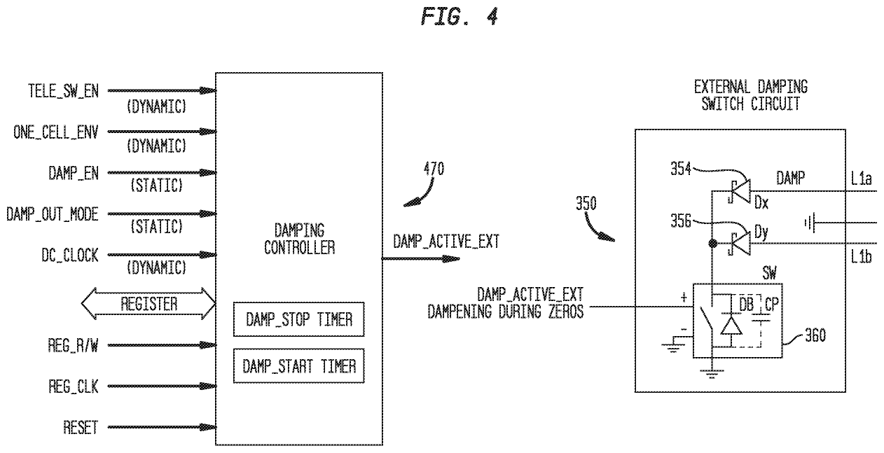

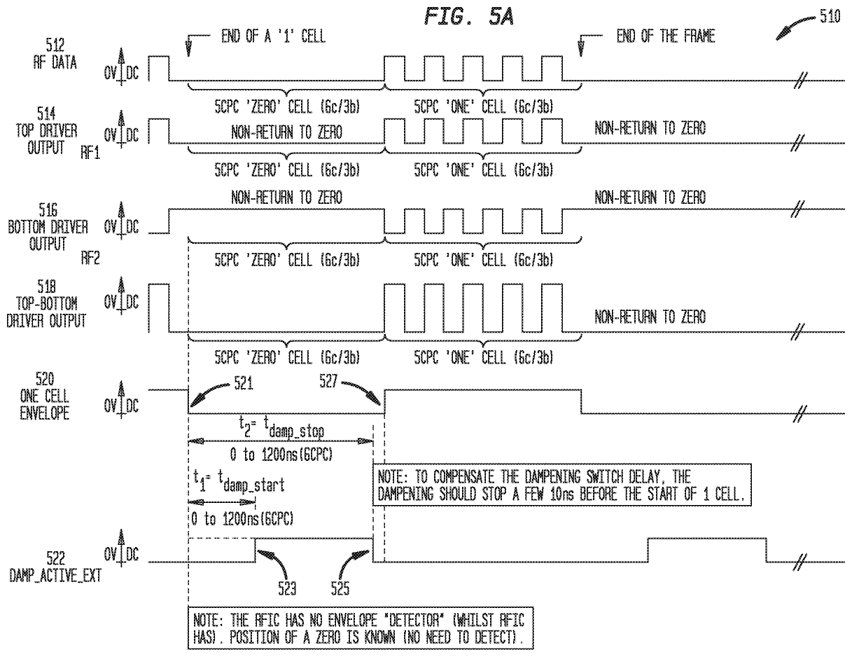

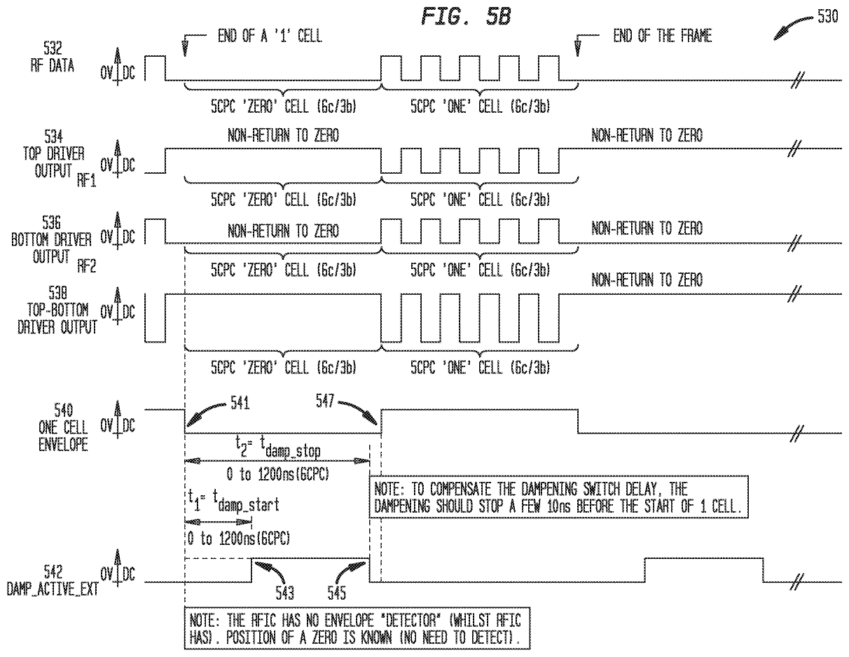

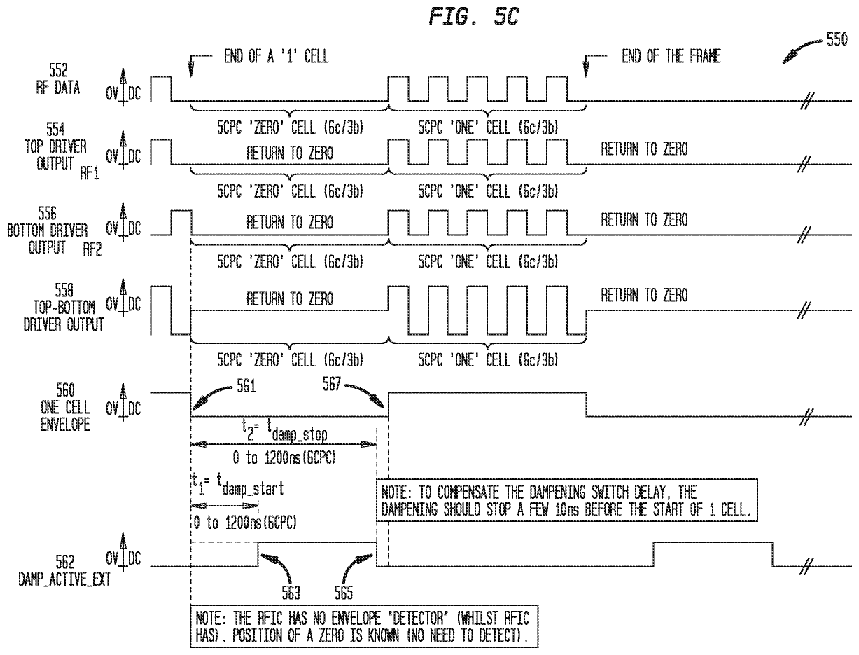

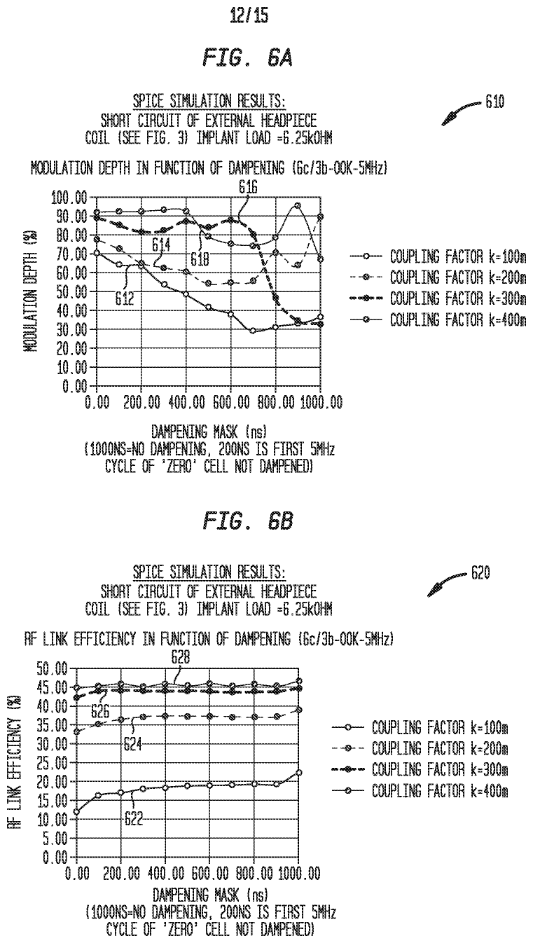

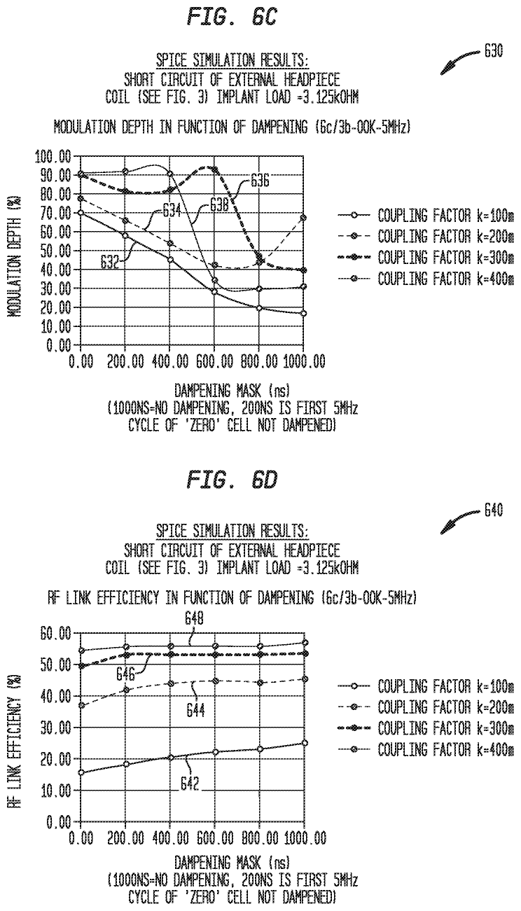

By introducing direct active damping, the stored energy in the resonant tank during a “zero cell” can be actively drained out of the resonant tank. This energy draining can be obtained by either one of the following possibilities: (1) active damping by adding a shunt resistance to the external coil (from the coupled resonant tank) using at least one external damping switch (e.g., one damping switch or two damping switches), as described below with reference to FIGS. 2A and 2B; or (2) active damping by electrical short circuit of the external coil inductance (from the coupled resonant tank) using an external damping switch (e.g., a single damping switch), as described below with reference to FIG. 3.

In accordance with certain embodiments, the single external coil can be connected with an external damping switch circuit including a damping switch (e.g., MOSFET) and a pair of diodes (e.g., Schottky diodes), wherein a damping controller controls an adjustable on-time of the damping switch to electrically short the external coil and thereby provide active damping, as described below with reference to FIG. 4.

As noted, by setting the on-time of the damping activation (e.g., straight after or after some delay from the end of the one cell, during the zero cell), the data integrity level (and damping strength) and the link efficiency can be adjusted. This adjusting of the strength of damping be achieved, for example, utilizing a programmable on-time of damping activation during the zero cells. In some example embodiments, the programmable on-time can result in the damping activation occurring after a short delay with respect to the end of a zero cell. Stated differently, the moment when the damping switch is closed can be controlled digitally according to the programmable on-time in order to delay the shorting to ground of the external coil. By delaying the shorting of the external coil, the damping strength (and thus the data integrity level) and the link efficiency can be adjusted, allowing more or less ringing in the resonant tank.

There are a number of different types of devices in/with which embodiments of the present invention can be implemented. Merely for ease of description, the techniques presented herein are primarily described with reference to a specific device in the form of a cochlear implant system. However, it is to be appreciated that the techniques presented herein can also be partially or fully implemented by any of a number of different types of devices, including consumer electronic devices (e.g., mobile phones), hearing devices, implantable medical devices, wearable devices (e.g., smart watches, etc.), etc. As used herein, the term “hearing device” is to be broadly construed as any device that delivers sound signals to a user in any form, including in the form of acoustical stimulation, mechanical stimulation, electrical stimulation, etc. As such, a hearing device can be a device for use by a hearing-impaired person (e.g., hearing aids, middle ear auditory prostheses, bone conduction devices, direct acoustic stimulators, electro-acoustic hearing prostheses, auditory brainstem stimulators, bimodal hearing prostheses, bilateral hearing prostheses, dedicated tinnitus therapy devices, tinnitus therapy device systems. combinations or variations thereof, etc.) or a device for use by a person with normal hearing (e.g., consumer devices that provide audio streaming, consumer headphones, earphones, and other listening devices). In other examples, the techniques presented herein can be implemented by, or used in conjunction with, various implantable medical devices, such as vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

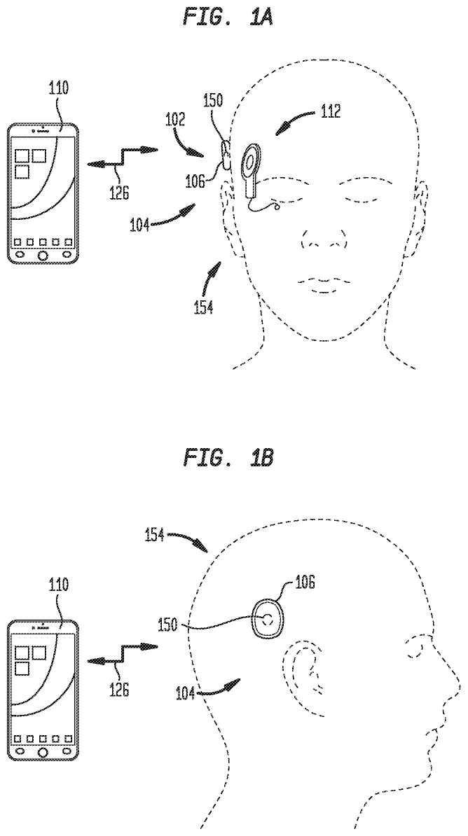

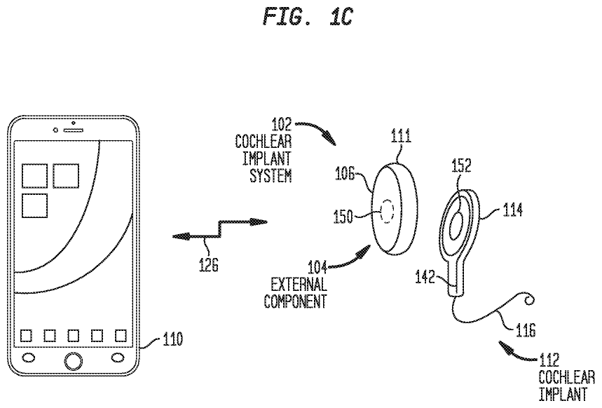

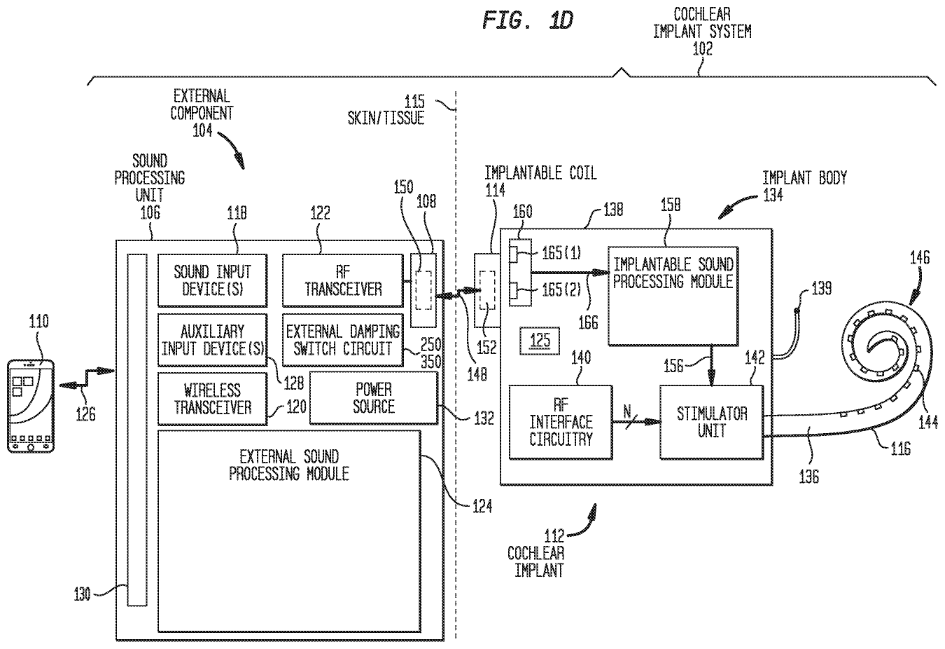

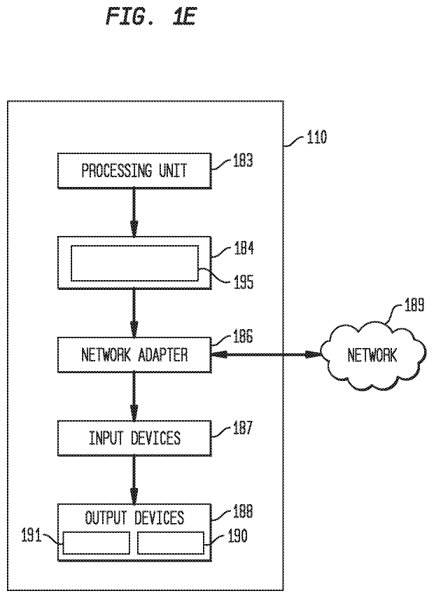

FIGS. 1A-1D illustrates an example cochlear implant system 102 with which aspects of the techniques presented herein can be implemented. The cochlear implant system 102 comprises an external component 104 that is configured to be directly or indirectly attached to the body of the user, and an internal/implantable component 112 that is configured to be implanted in or worn on the head of the user. In the examples of FIGS. 1A-1D, the implantable component 112 is sometimes referred to as a “cochlear implant.” FIG. 1A illustrates cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1D will generally be described together.

In the examples of FIGS. 1A-1D, the external component 104 comprises a sound processing unit 106, an external coil assembly 108, and generally, a magnet fixed relative to the external coil assembly 108. The cochlear implant 112 includes an implantable coil 114, an implant body 134, and an elongate stimulating assembly 116 configured to be implanted in the user's cochlea. In one example, the sound processing unit 106 is an off-the-ear (OTE) sound processing unit, sometimes referred to herein as an OTE component, which is configured to send data and power to the implantable component 112. In general, an OTE sound processing unit is a component having a generally cylindrically shaped housing 111 and which is configured to be magnetically coupled to the user's head 154 (e.g., includes an integrated external magnet 150 configured to be magnetically coupled to an internal/implantable magnet 152 in the implantable component 112). The OTE sound processing unit 106 also includes an integrated external (headpiece) coil (e.g., refer to external coil 240 of FIGS. 2 and 3, which can form a part of the external coil assembly 108) that is configured to be inductively coupled to the implantable coil 114.

It is to be appreciated that the OTE sound processing unit 106 is merely illustrative of the external devices that could operate with implantable component 112. For example, in alternative examples, the external component 104 can comprise a behind-the-ear (BTE) sound processing unit configured to be attached to, and worn adjacent to, the recipient's ear. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components could be located in the user's ear canal, worn on the body, etc.

Although the cochlear implant system 102 includes the sound processing unit 106 and the cochlear implant 112, as described below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110, which is shown in greater detail in FIG. IE, is a computing device, such as a personal computer (e.g., laptop, desktop, tablet), a mobile phone (e.g., smartphone), remote control unit, etc. The external device 110 and the cochlear implant system 102 (e.g., sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc.

Returning to the example of FIGS. 1A-ID, the sound processing unit 106 of the external component 104 also comprises one or more input devices configured to capture and/or receive input signals (e.g., sound or data signals) at the sound processing unit 106. The one or more input devices include, for example, one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a short-range wireless transmitter/receiver (wireless transceiver) 120 (e.g., for communication with the external device 110), each located in, on or near the sound processing unit 106. However, it is to be appreciated that one or more input devices can include additional types of input devices and/or less input devices (e.g., the short-range wireless transceiver 120 and/or one or more auxiliary input devices 128 could be omitted).

The sound processing unit 106 also comprises the external coil assembly 108, a charging coil 130, a closely-coupled radio frequency transmitter/receiver (RF transceiver) 122, an external damping switch circuit (e.g., refer to external damping switch circuit 250 of FIGS. 2A and 2B and/or external damping switch circuit 350 of FIG. 3), at least one power source 132 (e.g., a rechargeable battery, a replaceable battery, etc.), and an external sound processing module 124. The external coil assembly 108 can include one or more capacitors or capacitor arrays connected with the RF transceiver 122, and an external coil (headpiece coil) connected with the one or more capacitors/arrays and further connected with the external damping switch circuit (250/350). The external damping switch circuit can include, for example, one or more diodes (e.g., Schottky diodes), one or more damping switches (e.g., MOSFET), and (optionally) one or more resistors (e.g., shunt resistors) in series with the one or more diodes.

The external sound processing module 124 can be configured to perform a number of operations, and can be formed by one or more processors (e.g., one or more Digital Signal Processors (DSPs), one or more uC cores, etc.), firmware, software, etc. arranged to perform operations described herein. That is, the external sound processing module 124 can be implemented as firmware elements, partially or fully implemented with digital logic gates in one or more application-specific integrated circuits (ASICs), partially or fully in software, etc.

Returning to the example of FIGS. 1A-1D, the implantable component 112 comprises an implant body (main module) 134, a lead region 136, and the intra-cochlear stimulating assembly 116, all configured to be implanted under the skin (tissue) 115 of the user. The implant body 134 generally comprises a hermetically-sealed housing 138 that includes, in certain examples, at least one power source 125 (e.g., one or more batteries, one or more capacitors, etc.) 125, in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact array (electrode array) 146 for delivery of electrical stimulation (current) to the recipient's cochlea. Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. Implantable component 112 also includes an electrode outside of the cochlea, sometimes referred to as the extra-cochlear electrode (ECE) 139.

As noted, the cochlear implant system 102 includes the external coil assembly 108 and the implantable coil 114. The external magnet 150 is fixed relative to the external coil assembly 108 and the internal/implantable magnet 152 is fixed relative to the implantable coil 114. The external magnet 150 and the internal/implantable magnet 152 fixed relative to the external coil assembly 108 and the internal/implantable coil 114, respectively, facilitate the operational alignment of the external coil assembly 108 with the implantable coil 114. This operational alignment of the coils enables the external component 104 to transmit data and power to the implantable component 112 via a closely-coupled wireless link 148 formed between the external coil assembly 108 with the implantable coil 114. In certain examples, the closely-coupled wireless link 148 is a radio frequency (RF) link. However, various other types of energy transfer, such as infrared (IR), electromagnetic, capacitive, and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to process the received input audio signals (received at one or more of the input devices, such as sound input devices 118 and/or auxiliary input devices 128), and convert the received input audio signals into output control signals for use in stimulating a first ear of a recipient or user (i.e., the external sound processing module 124 is configured to perform sound processing on input signals received at the sound processing unit 106). Stated differently, the one or more processors (e.g., processing element(s) implementing firmware, software, etc.) in the external sound processing module 124 are configured to execute sound processing logic in memory to convert the received input audio signals into output control signals (stimulation signals) that represent electrical stimulation for delivery to the recipient.

As noted, FIG. 1D illustrates an embodiment in which the external sound processing module 124 in sound processing unit 106 generates the output control signals. In an alternative embodiment, the sound processing unit 106 can send less processed information (e.g., audio data) to the implantable component 112 and the sound processing operations (e.g., conversion of input sounds to output control signals 156) can be performed by a processor within the implantable component 112.

In FIG. 1D, according to an example embodiment, output control signals (stimulation signals) are provided to the RF transceiver 122, which transcutaneously transfers the output control signals (e.g., in an encoded manner) to the implantable component 112 via external coil assembly 108 and implantable coil 114. That is, the output control signals (stimulation signals) are received at the RF interface circuitry 140 via implantable coil 114 and provided to the stimulator unit 142. The stimulator unit 142 is configured to utilize the output control signals to generate electrical stimulation signals (e.g., current signals) for delivery to the user's cochlea via one or more of the stimulating contacts (electrodes) 144. In this way, cochlear implant system 102 electrically stimulates the user's auditory nerve cells, bypassing absent or defective hair cells that normally transduce acoustic vibrations into neural activity, in a manner that causes the recipient to perceive one or more components of the input audio signals (the received sound signals).

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, an example embodiment of the cochlear implant 112 can include a plurality of implantable sound sensors 165(1), 165(2) that collectively form a sensor array 160, and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 165(1), 165(2) of the sensor array 160 are configured to detect/capture input sound signals 166 (e.g., acoustic sound signals, vibrations, etc.), which are provided to the implantable sound processing module 158. The implantable sound processing module 158 is configured to convert received input sound signals 166 (received at one or more of the implantable sound sensors 165(1), 165(2)) into output control signals 156 for use in stimulating the first ear of a recipient or user (i.e., the implantable sound processing module 158 is configured to perform sound processing operations). Stated differently, the one or more processors (e.g., processing element(s) implementing firmware, software, etc.) in implantable sound processing module 158 are configured to execute sound processing logic in memory to convert the received input sound signals 166 into output control signals 156 that are provided to the stimulator unit 142. The stimulator unit 142 is configured to utilize the output control signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 165(1), 165(2) of sensor array 160 in generating stimulation signals for delivery to the user.

FIG. 1E is a block diagram illustrating one example arrangement for an external computing device 110 configured to perform one or more operations in accordance with certain embodiments presented herein. As shown in FIG. 1E, in its most basic configuration, the external computing device 110 includes at least one processing unit 183 and a memory 184. The processing unit 183 includes one or more hardware or software processors (e.g., Central Processing Units) that can obtain and execute instructions. The processing unit 183 can communicate with and control the performance of other components of the external computing device 110. Memory 184 is one or more software or hardware-based computer-readable storage media operable to store information accessible by the processing unit 183. Memory 184 can store, among other things, instructions executable by the processing unit 183 to implement applications or cause performance of operations described herein, as well as other data. The memory 184 can be volatile memory (e.g., RAM), non-volatile memory (e.g., ROM), or combinations thereof. Memory 184 can include transitory memory or non-transitory memory. The memory 184 can also include one or more removable or non-removable storage devices. In examples, the memory 184 can include random access memory (RAM), read only memory (ROM), EEPROM (Electronically-Erasable Programmable Read-Only Memory), flash memory, optical disc storage, magnetic storage, solid state storage, or any other memory media usable to store information for later access. By way of example, and not by limitation, memory 184 can include wired media such as a wired network or direct-wired connection, and wireless media such as acoustic, RF, infrared and other wireless media, or combinations thereof. In certain embodiments, memory 184 comprises logic 195 that, when executed, enables the processing unit 183 to perform aspects of the techniques presented.

In the illustrated example of FIG. 1E, the external computing device 110 further includes a network adapter 186, one or more input devices 187, and one or more output devices 188. The external computing device 110 can include other components, such as a system bus, component interfaces, a graphics system, a power source (e.g., a battery), among other components. The network adapter 186 is a component of the external computing device 110 that provides network access (e.g., access to at least one network 189). The network adapter 186 can provide wired or wireless network access and can support one or more of a variety of communication technologies and protocols, such as ETHERNET, cellular, BLUETOOTH, near-field communication, and RF (Radiofrequency), among others. The network adapter 186 can include one or more antennas and associated components configured for wireless communication according to one or more wireless communication technologies and protocols. The one or more input devices 187 are devices over which the external computing device 110 receives input from a user. The one or more input devices 187 can include physically-actuatable user-interface elements (e.g., buttons, switches, or dials), a keypad, keyboard, mouse, touchscreen, and voice input devices, among other input devices that can accept user input. The one or more output devices 188 are devices by which the computing device 110 is able to provide output to a user. The output devices 188 can include a display 190 (e.g., a liquid crystal display (LCD)) and one or more speakers 191, among other output devices for presentation of visual or audible information to the recipient, a clinician, an audiologist, or other user.

It is to be appreciated that the arrangement for the external computing device 110 shown in FIG. 1E is merely illustrative and that aspects of the techniques presented herein can be implemented at a number of different types of systems/devices including any combination of hardware, software, and/or firmware configured to perform the functions described herein. For example, the external computing device 110 can be a personal computer (e.g., a desktop or laptop computer), a hand-held device (e.g., a tablet computer), a mobile device (e.g., a smartphone), a surgical system, and/or any other electronic device having the capabilities to perform the associated operations described elsewhere herein.

As noted, communication between an external portion and an implant portion can occur over an inductively coupled link where the external coil and the implant coil are aligned, and both are part of a resonant tank. For data integrity purposes, damping is used during data transmission to distinguish one cells and zero cells in, for example, an On-Off Keying (OOK) modulation scheme. However, in conventional arrangements, when a one cell ends, the residual tank energy fades out but still can cause excessive ringing, and as a result, can hide a subsequent zero cell.

In operation, a cell can represent a binary value, or can belong to a group of cells that is translated into a set of binary values. For example, a 5 cycles-per-cell (5CPC) OOK scheme is one technique for sending ones and zeros over a radio frequency (RF) data link. In such examples, a “one cell” refers to 5 consecutive cycles of 200 ns each at 5 MHz in a 5 cycles-per-cell scheme, and a “zero cell” refers to a gap with a duration of 1 us (=5 cycles×200 ns) or 5 cycles at 5 MHz in a 5 cycles-per-cell scheme.

“Passive damping” can occur as a result of the quality factor (Q) drop from conductive losses of the drivers and the external coil copper losses. In contrast, “active damping” enforces extra damping on top of the passive damping. Utilizing active damping can improve data integrity on the implant system for combined RF links and split RF links. The goal of active damping is to remove the ringing during the zero cells. Active damping can result in a slightly lower RF link efficiency at higher SFT. In addition, damping requires extra size (more components) and/or has an extra PCB trace routing impact on the headpiece.

According to aspects presented herein, enhancement of data integrity for inductively coupled resonant tanks is obtained by selectively removing the energy from the sending resonant tank during zero cells, using a shorting switch directly to the inductor terminals within the resonant tank. Schottky diodes (Dx & Dy), for example, can be used to avoid conduction of the body diode of the MOSFET damping switch (SW). According to an example embodiment, this also allows the circuit to include a single damping switch. Additionally, resistors can be added (Rx & Ry), in which case the circuit can include one damping switch, or possibly two separate damping switches (e.g., to avoid an amplitude drop).

To provide DC free operation and to enhance the symmetry, a center tap (mid-tap) can optionally be added to the primary external coil, as shown in both FIGS. 2A and 2B and FIG. 3. If the grounding is placed in the mid-tap (i.e., between L1a/R1a and L1b/R1b), then there is no negative DC component (V=0 in center of HPC) and damping works normally.

If the grounding in the center/mid-tap is removed, there could potentially be an imbalance, depending on the construction of the coil. This is because a negative DC component on both coil ends would be created by rectification once the two damping Schottky diodes (Dx & Dy) of the external damping switch circuit become active by the closure of the damping switch (SW) during the zero cells, in which case the damping would not work properly anymore. Thus, the introduction of the mid-tap point allows the drivers to be driven differentially. For example, for a legacy monophasic driver scheme (varying the duty cycle), the top driver and the bottom driver need to be matched with the same duty cycle, in order not to create any unbalance.

FIGS. 2A and 2B are circuit diagrams for an external coil assembly with an external damping switch circuit coupled thereto, according to an example embodiment. FIGS. 2A and 2B are representations of active damping by adding shunt resistances (Rx & Ry) to the external coil using at least one external damping switch (e.g., one damping switch in FIG. 2A or two damping switches in FIG. 2B).

FIGS. 2A and 2B show a system 200 including an RF transceiver 122 (also referred to as a radio frequency integrated circuit, or RFIC), an external coil assembly 108 including an external coil 240 (part of a resonant tank), and an external damping switch circuit 250, according to an example embodiment. The RF transceiver 122 includes top drivers 212 and bottom drivers 218 configured to receive input signals (IN_x and IN_y) and transmit output signals (OUT_x and OUT_y, respectively).

The external coil assembly 108 includes one or more capacitors or capacitor arrays 232, 238 (e.g., C1x, . . . connected with the top drivers 212, and C1y, . . . connected with the bottom drivers 218). The capacitors/capacitor arrays 232, 238 can be utilized for external coil (HPC) tuning. External coil assembly 108 further includes a top coil 234 including an inductor 242 (L1a) and a resistor 244 (R1a), and a bottom coil 236 including an inductor 246 (L1b) and a resistor 248 (R1b). It is noted that the resistor 244 (R1a) and the resistor 248 (R1b) may not be physical resistors placed in the circuit, but rather may represent the equivalent series resistance of the inductor 242 (L1a) and the inductor 246 (L1b), respectively. The top coil 234 and the bottom coil 236 are collectively referred to herein as an external coil 240.

The external damping switch circuit 250 (Damp) includes two damping diodes 254, 256 (Dx & Dy), two shunt resistors 253, 257 (Rx & Ry), and at least one damping switch 260 (SW). The damping switch 260 can include a switch, and a body diode (Ds) and a capacitor (Cp) connected in parallel. In an example embodiment, the damping diodes 254, 256 can be Schottky diodes, and the damping switch 260 can be a MOSFET. Although only one damping switch 260 is shown in the external damping switch circuit 250 of FIG. 2A, there can be two ‘separated’ MOSFET damping switches in another example embodiment of the external damping switch circuit 250, as shown in FIG. 2B, in which a first damping switch 260-1 is connected to the damping diode 254 and a second damping switch 260-2 is connected to the damping diode 256, respectively, which can serve to avoid an amplitude drop that could potentially occur when one ‘common’ MOSFET damping switch is used.

The damping diode 254 (Dx) is connected in series with the shunt resistor 253 (Rx), which is further connected to the inductor terminal 233 of the top coil 234 of external coil 240 (i.e., an interconnection point between inductor 242 (L1a) and capacitor 232 (C1x)). The damping diode 256 (Dy) is connected in series with the shunt resistor 257 (Ry), which is further connected to the inductor terminal 237 of the bottom coil 236 of external coil 240 (i.e., an interconnection point between inductor 246 (L1b) and capacitor 238 (C1y)). The damping diodes 254, 256 are also connected with the damping switch 260 (SW).

In addition, a mid-tap 235 (or center tap) is added to the external coil 240, and is connected between the top coil 234 and the bottom coil 236 (i.e., the mid-tap 235 is added between the inductor 242 (L1a) and the inductor 246 (L1b) in FIGS. 2A and 2B). The mid-tap 235 is further connected to ground 255 via the external damping switch circuit 250. The mid-tap 235 is introduced between the top coil 234 and the bottom coil 236 (e.g., inductor 242 and inductor 246) to avoid any DC build up and/or avoid any asymmetry.

Thus, in the example embodiment of FIGS. 2A and 2B, the damping is performed using one damping switch or possibly two damping switches (e.g., MOSFETs), two diodes (e.g., Schottky diodes), and two resistors (e.g., shunt resistors). The external damping switch circuit 250 provides damping during zero cells by adding a shunt resistance (via shunt resistors 253, 257) to the external coil 240 (e.g., top coil 234 and bottom coil 236).

The solutions shown in FIGS. 2A and 2B support use of a single external coil in a combined RF link (e.g., modulated data with power) or a split RF link (e.g., power and data separated using a time division multiple access (TDMA) scheme), for example using quantized waveforms (QW). It is also noted that, in certain examples where magnetic induction (MI) is involved, using series resistors on each Schottky diode can provide for MI coexistence.

FIG. 3 is a circuit diagram for an external coil assembly with an external damping switch circuit coupled thereto, according to another example embodiment. FIG. 3 is a representation of active damping by electrical short of the external coil using an external switch.

FIG. 3 shows a system 300 including an RF transceiver 122 (RFIC), an external coil assembly 108 including an external coil 240 (part of a resonant tank), and an external damping switch circuit 350, according to another example embodiment. The RF transceiver 122 (including the top drivers 212 and the bottom drivers 218) and the external coil assembly 108 including the capacitors 232, 238 and the external coil 240 (including the top coil 234 and the bottom coil 236) are the same as described above with reference to FIGS. 2A and 2B.

The external damping switch circuit 350 (Damp) includes two damping diodes 354, 356 (Dx & Dy) and a damping switch 360 (SW). The damping switch 360 can include a switch, a body diode (Ds) and a capacitor (Cp) connected in parallel. In an example embodiment, the damping diodes 354, 356 can be Schottky diodes, and the damping switch 360 can be a MOSFET. Although only one damping switch 360 is shown in the external damping switch circuit 350 of FIG. 3, there can be two ‘separated’ MOSFET damping switches in another example embodiment of the external damping switch circuit 350 (similar to the example shown in FIG. 2B, but without the shunt resistors), in which a first damping switch is connected to the damping diode 254 and a second damping switch is connected to the damping diode 256, respectively, which can serve to avoid an amplitude drop that could potentially occur when one ‘common’ MOSFET damping switch is used.

The damping diode 354 (Dx) is connected to the inductor terminal 233 of the top coil 234 of external coil 240 (i.e., an interconnection point between inductor 242 (L1a) and capacitor 232 (C1x)). The damping diode 356 (Dy) is connected to the inductor terminal 237 of the bottom coil 236 of external coil 240 (i.e., an interconnection point between inductor 246 (L1b) and capacitor 238 (C1y)). The damping diodes 354, 356 are further connected with the damping switch 360 (SW). In the example embodiment of FIG. 3 (like in FIGS. 2A and 2B), the mid-tap 235 (center tap) is added between the top coil 234 and the bottom coil 236 of external coil 240, and is connected to ground 355 via the external damping switch circuit 350 to avoid any DC build up and/or avoid any asymmetry.

Thus, in the example embodiment of FIG. 3, the damping is performed using one damping switch (e.g., MOSFET) and two diodes (e.g., Schottky diodes). The external damping switch circuit 350 provides damping during zero cells by shorting the external coil 240 (e.g., top coil 234 and bottom coil 236).

The solution shown in FIG. 3 supports use of a single external coil in a combined RF link (e.g., modulated data with power) or a split RF link (e.g., power and data separated using a time division multiple access (TDMA) scheme), for example using quantized waveforms (QW).

The external damping switch circuit 350 of FIG. 3 is similar to the external damping switch circuit 250 of FIGS. 2A and 2B, with one exception-the external damping switch circuit 350 of FIG. 3 does not include shunt resistors (Rx & Ry). In certain examples, the shunt resistors 253, 257 (refer to FIGS. 2A and 2B) can add robustness for magnetic induction (MI), where an MI link is used in addition to the RF link. If there is no MI link or MI is not used, however, then the shunt resistors can be omitted from the external damping switch circuit 350 (refer to FIG. 3) to reduce the size and number of components of the external damping switch circuit (Damp).

FIGS. 2A and 2B and FIG. 3 illustrate the multiplicity of drivers integrated in the RF transceiver 122 (RFIC), and show distributed balanced capacitors 232, 238 at the output of the different drivers 212, 218. It is to be appreciated there are different possibilities to apply the tuning of the external coil 240, and there can be only a single tuning capacitor in the external coil assembly 108 according to an alternative example embodiment.

FIG. 4 is a circuit diagram showing a damping controller 470 that is configured to control the external damping switch circuit, in accordance with certain embodiments presented herein. For ease of description, the damping controller 470 is described with reference to control of the external damping switch circuit 350 (e.g., damping switch 360) of FIG. 3. As such, although not shown in FIG. 4, the damping controller 470 could be configured to control the external damping switch circuit 250 (e.g., damping switch 260) of FIGS. 2A and 2B in a similar manner.

Damping of zero cells occurs in the absence of the one cells (ONE_CELL_ENV=0) and in the absence of the telemetry signaling (TELE_SW_EN=0). The hardware implementation defines the setting of the DAMP_OUT_MODE. The selection for an external damping output signal (DAMP_ACTIVE_EXT) for driving the external damping switch circuit 350 (damping switch 360) requires DAMP_OUT_MODE=1. Otherwise, the internal damping ground switches (L1_1, L1_2) would be driven by a DAMP_SW_INT signal (not shown).

The rising edges of the damping output signal (DAMP_ACTIVE_EXT) are delayed between 0 ns and 1/f (where f=2.5 MHz, 5 MHz, or 6.78 MHz)×CPC (where CPC=4, 5, or 6) by the damping controller 370, with a resolution of at least 6 bits (although the resolution could be less than or greater than this example). The programmable on-time of activation of the damping switch is adjustable to allow the damping force to be scaled appropriately.

By utilizing a programmable on-time that can be set and adjusted according to techniques described herein, it is possible to delay the damping “ON” feature in steps of 100 ns, or more preferably, steps of 20 ns, through a register setting (DAMP_START) for the damping controller 470. The damping start (programmable on-time) can be set such that damping activation occurs after some delay with respect to the end of a one cell when the external damping switch circuit 250 (damping switch 260) or the external damping switch circuit 350 (damping switch 360) is utilized, with a selected step size (e.g., 20 ns, 100 ns, etc.). Likewise, by utilizing a programmable off-time that can be set and adjusted according to techniques described herein, it is possible to delay the damping “OFF” feature in steps of 100 ns, or more preferably, steps of 20 ns, through a register setting (DAMP_STOP) for the damping controller 470. The damping stop (programmable off-time) can be set such that damping deactivation occurs shortly before the beginning of the next one cell when the external damping switch circuit 250 (damping switch 260) or the external damping switch circuit 350 (damping switch 360) is utilized, with a selected step size (e.g., 20 ns, etc.). In certain examples, the damping duration (damping active time) is represented by the following equation: DAMP_ACTIVE_EXT=DAMP_STOP−DAMP_START.

FIG. 5A is a timing diagram 510 showing a 5CPC RF data stream for a monophasic driver output signal (for legacy devices). The timing diagram 510 shows timing (0=low and 1=high) for the following signals: RF data 512, top driver output 514 (RF1), bottom driver output 516 (RF2), top-bottom driver output 518, one cell envelope 520, and a damping duration (damping active time) 522 (DAMP_ACTIVE_EXT).

The one cell envelope 520 includes an end of a first one cell 521 (transition from 1=high to 0=low), from which t₁ (t_{damp_start}) and t₂ (t_{damp_stop}) begin, where t₁ and t₂ are programmable and adjustable values between Ons and 1200 ns, for example (although this is dependent on and may vary according to the chosen number CPC and the frequency used in other examples). After t₁ (t_{damp_start}) elapses, the damping turns ON 523 (1=high). After t₂ (t_{damp_stop}) elapses, the damping turns OFF 525 (0=low). To compensate for the damping switch delay, the damping should stop a few 10 ns before the start of a second one cell 527 (transition from 0=low to 1=high) of the one cell envelope 520.

FIG. 5B is a timing diagram 530 showing a 5CPC RF data stream for an inverted monophasic driver output signal. The timing diagram 530 shows timing (0=low and 1=high) for the following signals: RF data 532, top driver output 534 (RF1), bottom driver output 536 (RF2), top-bottom driver output 538, one cell envelope 540, and a damping duration (damping active time) 542 (DAMP_ACTIVE_EXT).

The one cell envelope 540 includes an end of a first one cell 541 (transition from 1=high to 0=low), from which t₁ (t_{damp_start}) and t₂ (t_{damp_stop}) begin, where t₁ and t₂ are programmable and adjustable values between Ons and 1200 ns, for example (although this may depend on and may vary according to the chosen number CPC and the frequency used in other examples). After t₁ (t_{damp_start}) elapses, the damping turns ON 543 (1=high). After t₂ (t_{damp_stop}) elapses, the damping turns OFF 545 (0=low). To compensate for the damping switch delay, the damping should stop a few 10 ns before the start of a second one cell 547 (transition from 0=low to 1=high) of the one cell envelope 540.

FIG. 5C is a timing diagram 550 showing a 5CPC RF data stream for a biphasic driver output signal. The timing diagram 550 shows timing (0=low and 1=high) for the following signals: RF data 552, top driver output 554 (RF1), bottom driver output 556 (RF2), top-bottom driver output 558, one cell envelope 560, and a damping duration (damping active time) 562 (DAMP_ACTIVE_EXT).

The one cell envelope 560 includes an end of a first one cell 561 (transition from 1=high to 0=low), from which t₁ (t_{damp_start}) and t₂ (t_{damp_stop}) begin, where t₁ and t₂ are programmable and adjustable values between Ons and 1200 ns, for example (although this may depend on and may vary according to the chosen number CPC and the frequency used in other examples). After t₁ (t_{damp_start}) elapses, the damping turns ON 563 (1=high). After t₂ (t_{damp_stop}) elapses, the damping turns OFF 565 (0=low). To compensate for the damping switch delay, the damping should stop a few 10 ns before the start of a second one cell 567 (transition from 0=low to 1=high) of the one cell envelope 560.

In some example embodiments, the damping feature operates during a maximum of two zero cells and next to the frame end for 5 MHz links at 4CPC, 5CPC, and 6CPC. The damping feature can be enabled/disabled at the end of each RF data frame, or at the end of each power frame (5 MHz or 6.78 MHz).

As noted above, the active damping of the external coil can occur with a selectable/programmable delay relative to the end of a one cell, according to the programmable on-time (DAMP_START). That is, the moment of activation of the damping can be set to, for example, optimize the data integrity level (and damping strength) and the link efficiency. In accordance with certain embodiments presented herein, the programmable on-time (and hence, the adjustable delay with respect to the end of a one cell) for the damping activation can be set (or adjusted) based on a coupling factor (magnetic coupling factor (k)) of the external coil (transmitting coil) and an implantable coil (receiving coil). The coupling factor could be affected, for example, by a distance between the external coil and the implantable coil, when in operation. The thickness of the skin flap (e.g., skin, tissue, etc.) of the particular recipient affects the distance between the external coil and the implantable coil and, in turn, the coupling factor.

The programmable on-time (DAMP_START) can be set or adjusted, via the damping controller 470 of FIG. 4, to control or modify modulation depth (e.g., to increase or maximize the modulation depth). The “ringing” phenomenon may depend, at least in part, on the coupling coefficient between the external coil and the implantable coil. Depending on when the damping activation starts, the modulation depth can be increased. In some examples, the programmable on-time DAMP_START (and hence the delay of damping activation after the end of a one cell) may be set based on the coupling factor between the external and implantable coils. In some other examples, the programmable on-time DAMP_START (and hence the delay of damping activation after the end of a one cell) may be set based on the skin-flap thickness. The modulation depth is a function of the damping start time (or the damping delay with respect to the end of a one cell). For example, it may be desirable to start the damping activation sooner (by setting an earlier on-time, resulting in less delay) in the case of looser/farther coupling, or start the damping activation later (by setting a later on-time, resulting in more delay) in the case of closer coupling. FIG. 6A shows graphical simulation results 610 plotting modulation depth as a function of damping active time for various coupling factors (k), with an implant load of 6.25 kOhm. The graphical simulation results 610 show a curve 612 for a coupling factor (k=100 m), a curve 614 for a coupling factor (k=200 m), a curve 616 for a coupling factor (k=300 m), and a curve 618 for a coupling factor (k=400 m).

FIG. 6B shows graphical simulation results 620 plotting RF link efficiency as a function of damping active time for various coupling factors (k), with an implant load of 6.25 kOhm. The graphical simulation results 620 show a curve 622 for a coupling factor (k=100 m), a curve 624 for a coupling factor (k=200 m), a curve 626 for a coupling factor (k=300 m), and a curve 628 for a coupling factor (k=400 m).

FIG. 6C shows graphical simulation results 630 plotting modulation depth as a function of damping active time for various coupling factors (k), with an implant load of 3.125 kOhm. The graphical simulation results 630 show a curve 632 for a coupling factor (k=100 m), a curve 634 for a coupling factor (k=200 m), a curve 636 for a coupling factor (k=300 m), and a curve 638 for a coupling factor (k=400 m).

FIG. 6D shows graphical simulation results 640 plotting RF link efficiency as a function of damping active time for various coupling factors (k), with an implant load of 3.125 kOhm. The graphical simulation results 640 show a curve 642 for a coupling factor (k=100 m), a curve 644 for a coupling factor (k=200 m), a curve 646 for a coupling factor (k=300 m), and a curve 648 for a coupling factor (k=400 m).

As described above, the damping duration during the zero cells is made programmable (in steps of 100 ns, or preferably steps of 20 ns). In one example, a damping duration of 200 ns means that the first cycle is not dampened.

As shown in FIGS. 6A-6D, the modulation depth and the RF link efficiency vary depending on the magnetic coupling factor (k). From the graphical simulation results 610 and 630, it is observed that the ‘damping active time’ controls the modulation depth well, especially on lower coupling factors (e.g., k=100 m, k=200 m). From the graphical simulation results 620 and 640, it is advised to start the damping only after 100 ns next to the end of the ‘1 cycle’ or the end of the ‘one cell envelope’ to improve RF link efficiency at lower coupling factors (e.g., k=100 m, k=200 m).

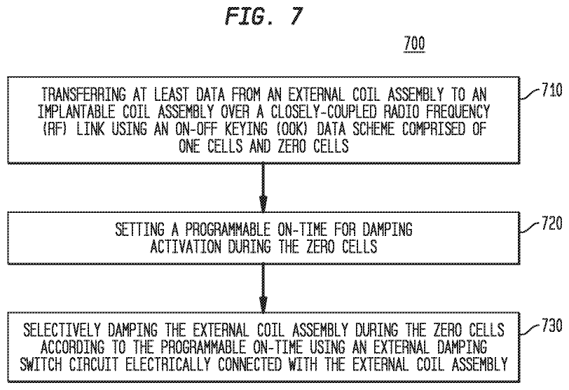

FIG. 7 is a flowchart of a method 700 for direct active damping with adjustable strength, according to an example embodiment. At operation 710, the method 700 includes transferring at least data from an external coil assembly to an implantable coil assembly over a closely-coupled radio frequency (RF) link using an on-off keying (OOK) data scheme comprised of one cells and zero cells. At operation 720, the method 700 includes setting a programmable on-time for damping activation during the zero cells. At operation 730, the method 700 includes selectively damping the external coil assembly during the zero cells according to the programmable on-time using an external damping switch circuit electrically connected with the external coil assembly.

In some examples, the external damping switch circuit is controlled using a damping controller electrically connected with the external damping switch circuit to selectively damp the external coil assembly during the zero cells according to the programmable on-time based on a coupling factor between the external coil assembly and the implantable coil assembly. In some examples, the external coil assembly is separated from the implantable coil assembly by a skin flap, the coupling factor varies based on a thickness of the skin flap, and the programmable on-time can be set based on the thickness of the skin flap or based on the coupling factor.

In some examples, the programmable on-time for the damping activation during the zero cells may be set or adjusted to modify (e.g., increase or maximize) at least one of a data integrity level (damping strength) or a link efficiency of the closely-coupled RF link. In some examples, the programmable on-time for the damping activation during the zero cells may be set or adjusted to modify (e.g., increase or maximize) a modulation depth associated with transmission of the data on the closely-coupled RF link. In some examples, the programmable on-time (DAMP_START) may be set or adjusted in steps of 100 ns, or more preferably, in steps of 20 ns.

In some examples, selectively damping the external coil assembly comprises selectively adding at least one shunt resistance to the external coil assembly via the external damping switch circuit. In some examples, selectively damping the external coil assembly comprises selectively shorting the external coil assembly via the external damping switch circuit.

In some examples, the external damping switch circuit is electrically connected with the external coil assembly, and is controlled via a damping controller electrically connected with the external damping switch circuit according to an adjustable delay setting for damping activation during the zero cells starting from an end of the one cells (via the programmable on-time), to provide selective direct active damping of the transmission of the data on the closely-coupled RF link by shorting the external coil during the zero cells, such that stored energy in the external coil is actively drained out of the external coil during the zero cells via the external damping switch circuit.

Thus, the systems and techniques described above with reference to FIGS. 2A, 2B, 3, 4, 5A-5C, 6A-6D, and 7 enable a wireless signal to be transferred over closely coupled resonant tanks of an AIMD system, containing a series of one cells and zero cells following an on-off keying (OOK) data scheme where a one cell has a predefined number of cycles and a zero cells has a predefined number of absent cycles, wherein damping of the wireless signal occurs during zero cells, with an adjustable delay setting in the damping activation (according to the programmable on-time), starting from the end of each one cell. This provides damping for both the combined RF link and the split RF link.

In one example embodiment, the one cells provide energy to the implant. This provides damping for the combined RF link, specifically.

The systems 200 and 300 described above with reference to FIGS. 2A, 2B, 3, and 4 provide an external coil assembly (e.g., including an external coil 240) and an external portion inductively coupled to the implant coil of an implantable portion of an AIMD system including an external damping circuit (e.g., external damping switch circuit 250 and external damping switch circuit 350) with a damping controller (e.g., damping controller 470). The damping controller uses a programmable on-time (DAMP_START) to enable an adjustable delay setting in the damping activation, for the purpose of reliable data transfer.

The external coil assembly (external coil) is part of a “series” LC resonant tank with inductor L. The damping circuit is electrically connected to the inductor L (i.e., the damping circuit is directly connected to the L and C interconnection point within the resonant tank).

In an example embodiment, the damping circuit consists of an electrical shorting switch (e.g., MOSFET damping switch 360) with series diodes (e.g., Schottky damping diodes 354, 356) to prevent DC build up during damping (FIG. 3).

In an example embodiment, the damping circuit consists of an electrical shorting switch (e.g., MOSFET damping switch 260) with series diodes (e.g., Schottky damping diodes 254, 256) to prevent DC build up during damping (e.g., see FIG. 2A), and also includes additional resistors (e.g., shunt resistors 253, 257).

An adapted inductor (e.g., top coil 234 with inductor 242 (L1a), and bottom coil 236 with inductor 246 (L1b)) can be used to enhance symmetry and prevent DC build up, by introducing a mid-tapped coil (mid-tap 235 of external coil 240), with the mid-tap connected to the ground node (via the external damping switch circuit 250 or the external damping switch circuit 350).

The solutions described herein do not change the current way of driving the external coil. During both one cells and zero cells, the driver keeps driving a one or a zero, respectively. Rather, the solutions described herein change the way of extracting the energy from the resonant tank (which causes the ringing). The remaining energy in the resonant tank is removed by directly interfacing with the external coil, rather than using an additional coupled coil to extract the remaining energy.

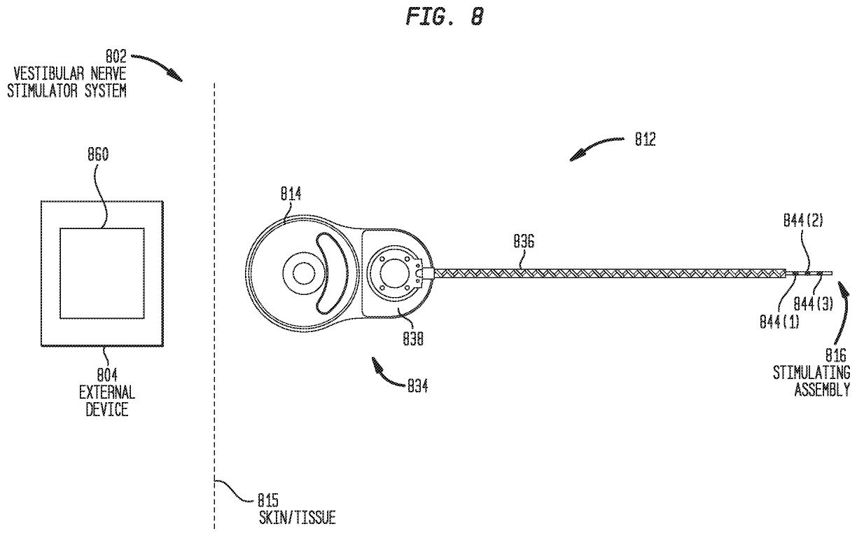

As previously described, the technology disclosed herein can be applied in any of a variety of circumstances and with a variety of different devices. Example devices that can benefit from technology disclosed herein are described in more detail in FIG. 8. The techniques of the present disclosure can be applied to other devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, the techniques described herein can also be applied to consumer devices. These different systems and devices can benefit from the technology described herein.

FIG. 8 illustrates an example vestibular stimulator system 802, with which embodiments presented herein can be implemented. As shown, the vestibular stimulator system 802 comprises an implantable component (vestibular stimulator) 812 and an external device 804 (e.g., external processing device, battery charger, remote control, etc.). The external device 804 comprises a transceiver unit 860. As such, the external device 804 is configured to transfer data (and potentially power) to the vestibular stimulator 812.

The vestibular stimulator 812 comprises an implant body (main module) 834, a lead region 836, and a stimulating assembly 816, all configured to be implanted under the skin/tissue (tissue) 815 of the recipient. The implant body 834 generally comprises a hermetically-sealed housing 838 in which RF interface circuitry, one or more rechargeable batteries, one or more processors, and a stimulator unit are disposed. The implant body 834 also includes an internal/implantable coil 814 that is generally external to the housing 838, but which is connected to the transceiver via a hermetic feedthrough (not shown).

The stimulating assembly 816 comprises a plurality of electrodes 844(1)-(3) disposed in a carrier member (e.g., a flexible silicone body). In this specific example, the stimulating assembly 816 comprises three (3) stimulation electrodes, referred to as stimulation electrodes 844(1), 844(2), and 844(3). The stimulation electrodes 844(1), 844(2), and 844(3) function as an electrical interface for delivery of electrical stimulation signals to the recipient's vestibular system.

The stimulating assembly 816 is configured such that a surgeon can implant the stimulating assembly adjacent to the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 812, the external device 804, and/or another external device, can be configured to implement the techniques presented herein. That is, the vestibular stimulator 812, possibly in combination with the external device 804 and/or another external device, can include an evoked biological response analysis system, as described elsewhere herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A device, comprising:
a radio frequency (RF) transceiver;
an external coil assembly including an external coil configured to be inductively coupled with an implantable coil to form a closely-coupled RF link; and
an external damping switch circuit electrically connected with the external coil, wherein the external damping switch circuit is controlled according to a programmable on-time for damping activation, to provide selective active damping of the external coil during transmission of data on the closely-coupled RF link.

2. The device of claim 1, wherein the external damping switch circuit is configured to selectively add at least one shunt resistance to the external coil to provide the selective active damping of the external coil.

3. The device of claim 1, wherein the external damping switch circuit is configured to selectively short the external coil to provide the selective active damping of the external coil.

4. The device of claim 1, wherein the external damping switch circuit is controllable to damp the external coil during zero cells of an on-off keying (OOK) data scheme such that stored energy in the external coil is actively drained out of the external coil during the zero cells via the external damping switch circuit.

5. The device of claim 4, wherein the OOK data scheme includes one cells, and wherein the one cells of the OOK data scheme provide data and power to the implantable coil via the closely-coupled RF link.

6. The device of claim 4, wherein the OOK data scheme includes one cells, and wherein the one cells of the OOK data scheme provide only data to the implantable coil via the closely-coupled RF link.

7. The device of claim 4, further comprising:
a damping controller connected with the external damping switch circuit, wherein the damping controller is configured to control the external damping switch circuit according to the programmable on-time for damping activation during the zero cells.

8. The device of claim 7, wherein the damping controller is configured to set the programmable on-time to modify at least one of a data integrity level or a link efficiency of the closely-coupled RF link.

9. The device of claim 7, wherein the damping controller is configured to set the programmable on-time based on at least one of a coupling factor between the external coil and the implantable coil or a modulation depth associated with transmission of the data on the closely-coupled RF link.

10. The device of claim 7, wherein the implantable coil is separated from the external coil by a skin flap, and wherein the damping controller is configured to set the programmable on-time based on a thickness of the skin flap.

11. (canceled)

12. (canceled)

13. The device of claim 1, wherein the RF transceiver comprises one or more first drivers and one or more second drivers, wherein the one or more first drivers and the one or more second drivers are connected to opposing ends of the external coil.

14. (canceled)

15. The device of claim 1, wherein the external damping switch circuit includes a first damping diode, a second damping diode, and at least one damping switch connected with the first damping diode and the second damping diode.

16. (canceled)

17. (canceled)

18. (canceled)

19. The device of claim 1, wherein the external coil assembly comprises:
a first inductor;
a first capacitor connected between the RF transceiver and the first inductor;
a second inductor; and
a second capacitor connected between the RF transceiver and the second inductor,
wherein the first inductor is connected in series with the second inductor, and wherein the external coil further includes a mid-tap between the first inductor and the second inductor of the external coil assembly, wherein the mid-tap is connected to ground via the external damping switch circuit.

20. (canceled)

21-36. (canceled)

37. A method comprising:
transferring at least data from an external coil assembly to an implantable coil assembly over a closely-coupled radio frequency (RF) link using an on-off keying (OOK) data scheme comprised of one cells and zero cells;
setting a programmable on-time for damping activation during the zero cells; and
selectively damping the external coil assembly during the zero cells according to the programmable on-time using an external damping switch circuit electrically connected with the external coil assembly.

38. The method of claim 37, further comprising:
controlling the external damping switch circuit using a damping controller to selectively damp the external coil assembly during the zero cells according to the programmable on-time based on a coupling factor between the external coil assembly and the implantable coil assembly.

39. The method of claim 38, wherein the external coil assembly is separated from the implantable coil assembly by a skin flap, the coupling factor varies based on a thickness of the skin flap, and the programmable on-time is set based on the thickness of the skin flap.

40. The method of claim 38, further comprising:
adjusting the programmable on-time for damping activation during the zero cells to modify at least one of a data integrity level, a link efficiency of the closely-coupled RF link, or a modulation depth associated with transmission of the data on the closely-coupled RF link

41. (canceled)

42. (canceled)

43. The method of claim 37, wherein selectively damping the external coil assembly comprises selectively adding at least one shunt resistance to the external coil assembly via the external damping switch circuit.

44. The method of claim 37, wherein selectively damping the external coil assembly comprises selectively shorting the external coil assembly via the external damping switch circuit.

45. The method of claim 37, wherein selectively damping the external coil assembly actively drains out stored energy in the external coil assembly during the zero cells via the external damping switch circuit.
